(19) United States
(12) Patent Application Publication
(10) Pub. No.: US 2017/0007837 A1
(43) Pub. Date: Jan. 12, 2017

(54) SYSTEMS AND METHODS FOR ANCHORING PRE-MOLDED HEADER CONNECTOR ASSEMBLY TO HOUSING OF IMPLANTABLE PULSE GENERATOR

(71) Applicant: PACESETTER, INC., Sylmar, CA (US)

(72) Inventors: Asghar Dadashian, Porter Ranch, CA (US); Ke Zeng, South Pasadena, CA (US); Avi Bilu, Pasadena, CA (US); Mitch Goodman, Santa Clarita, CA (US)

(21) Appl. No.: 14/794,169

(22) Filed: Jul. 8, 2015

(51) Int. Cl. A61N 1/375

(52) U.S. Cl. CPC A61N 1/3752

(57) ABSTRACT

Disclosed herein is an implantable pulse generator for administering electrotherapy via an implantable lead. The pulse generator includes a housing and a header connector assembly. The housing includes a first portion of a cam lock arrangement. The header connector assembly includes a connector assembly, a header enclosing the connector assembly, and a second portion of the cam lock system cam locked with the first portion in anchoring the header connector assembly to the housing.

### FIELD OF THE INVENTION

Aspects of the present invention relate to medical apparatus and methods. More specifically, the present invention relates to systems and methods for anchoring a pre-molded header connector assembly to a housing of an implantable pulse generator.

### BACKGROUND OF THE INVENTION

An Implantable pulse generators (IPGs) such as pacemakers and implantable cardioverter defibrillators (ICDs), which are used in the treatment of cardiac conditions, and neuromodulators or neurostimulators, which are used in chronic pain management or the actuation and control of other body systems, commonly include a housing, feedthrus, and a connector assembly that is enclosed in a header. Electrical stimulation originating in the housing is led to the connector assembly through feedthrus. The connector assembly serves to transmit electrical signals out of the IPG and to a lead electrically connected to the connector assembly, the lead transmitting electrical signals between the IPG and patient tissue.

Current header casting manufacturing processes and the associated methods of assembling the header and its enclosed connector assembly onto the housing require multiple operations, are skill intensive, and unavoidably time consuming. Connector assemblies are first cast into a header separate from the housing, the header and the connector assembly enclosed therein forming a header connector assembly. The header connector assembly is joined with the housing by injecting a thermosetting polymer (e.g., an epoxy) into an interface between the header connector assembly and the housing, such an injection process being called a backfill process. This backfill process creates attachment and electrical sealing between the header connector assembly and the housing. However, the backfill process nearly mirrors the extensive casting process used to encase the connector assembly in the header to form the header connector assembly, the backfill process involving mold set-up, mold pre-heat, epoxy dispense, epoxy curing, and mold breakdown. The backfill process is not only lengthy, but also expensive due to its many tools and equipment, and necessity for many skilled operators.

Due to the low viscosity characteristics of epoxy used in the backfill process, the epoxy has a tendency to flow into undesired areas. A common cause for rework on IPGs involves epoxy entering one or more of the lead connector receiving bores of the header connector assembly, thereby forming a barrier to the establishment of critical electrical connections between the electrical terminals of the lead connector ends and the electrical contacts of the connector assembly. Such IPG rework further extends costs and manufacturing times. Other causes for rework are experienced throughout the casting and backfill processes.

There is a need in the art for systems and methods that eliminate the backfill process.

### BRIEF SUMMARY OF THE INVENTION

The anchoring arrangements and methods disclosed herein allow for the elimination of the backfill process. In one embodiment, mechanical attachment and compression between two IPG modules (e.g., a header connector assembly and a housing) is established through a cam and pin arrangement. In another embodiment, mechanical attachment and compression between the two IPG modules is established through a welded arrangement. Either of these anchoring mechanisms can be used to eliminate the process of backfilling with epoxy, which has traditionally been the method of attaching these two IPG modules. Once the modules are anchored via either of these two mechanisms, sealing can be achieved through medical adhesive application, which is a much quicker and simpler process in comparison to backfill.

An additional advantage of the anchoring arrangements and methods disclosed herein is compression through mechanical fastening creates pre-load between the two IPG models, which is important to an IPG's longevity. Due to pre-load, a force or torque that is equal or greater than the pre-load must be applied before there is even slight movement between the modules. Thus, pre-load reduces fatigue caused by cyclic loading and increases torque and force capacity between the two IPG modules. Delamination and adhesive detachment are often caused by movement between the IPG modules, and the reduction of delamination or adhesive detachment reduces the likelihood of leakage or other IPG failure.

The cam and pin arrangement includes access points that expose connector pins where they are in contact with a cam connector, permitting welding to prevent anchor loosening. Adhesive can be injected through the access points to secure the attachment. It can provide strong adhesion due to high contact area between the connector pin and cam connector. Additionally cured adhesive interlocking within the cam and pin arrangement prevents dislodging.

The backfill attachment method known in the art for attaching the header connector assembly to the housing of an IPG requires a material that can bond epoxy to titanium. Both the cam and pin arrangement and the welded arrangement disclosed herein are independent of the materials used in constructing the header connector assembly and housing. Thus, these anchoring arrangements can secure the header connector assembly to the housing regardless of construction materials of these two IPG modules. Also, both of these anchoring arrangements are space saving and require little space to implement.

One implementation of the present disclosure may take the form of an implantable pulse generator for administering electrotherapy via an implantable lead. The pulse generator includes a housing and a header connector assembly. The housing includes a first portion of a cam lock arrangement. The header connector assembly includes a connector assembly, a header enclosing the connector assembly, and a second portion of the cam lock system cam locked with the first portion in anchoring the header connector assembly to the housing.

In one embodiment, the first portion may include a pin and the second portion may include a cam that is cam locked with the pin. The pin may be a pair of spaced-apart pins, and the cam may be a cam that is cam locked with the pair of spaced-apart pins. The cam will have been rotated to become cam locked with the at least one pin. Additionally, the cam may be both cam locked and welded with the pin. The cam lock arrangement compresses together the header connector assembly and the housing.

There may be three pairs of spaced-apart pins and three cams that are spaced-apart from each other, each cam being cam locked with a respective one of the three pairs of spaced-apart pins.

The pin may be defined in a material forming the housing. Alternatively, the pin may be welded to the housing prior to the housing having been hermetically sealed or certified.

In another embodiment, the second portion includes a pin and the first portion includes a cam that is cam locked with the pin.

Another implementation of the present disclosure may take the form of an implantable pulse generator for administering electrotherapy via an implantable lead. The pulse generator includes a housing and a header connector assembly. The housing includes a weld anchor. The header connector assembly includes a connector assembly, a header enclosing the connector assembly, and a weld insert imbedded in a material of the header and welded to the weld anchor in anchoring the header connector assembly to the housing.

The weld insert may include three weld inserts spaced-apart from each other, and the weld anchor may include two pairs of spaced-apart weld anchors. Each pair of spaced-apart weld anchors is paired with two of the weld inserts, and the third weld insert is welded to an RF anchor of the connector assembly.

The weld anchor may be defined in a material forming the housing. Alternatively, the weld anchor may be welded to the housing prior to the housing having been hermetically sealed or certified.

Yet another implementation of the present disclosure may take the form of a method of manufacturing an implantable pulse generator for administering electrotherapy via an implantable lead The method includes: abutting together a housing and a pre-manufactured header connector assembly, the housing comprising a first portion of a cam lock arrangement, and the pre-manufactured header connector assembly comprising a connector assembly, a header enclosing the connector assembly, and a second portion of the cam lock arrangement; and cam locking together the first and second portions of the cam lock arrangement.

The method may further include applying a compressive pre-load to the housing and pre-manufactured header connector assembly in abutting together the housing and pre-manufactured header connector assembly, the compressive pre-load being in existence prior to and during the cam locking together the first and second portions of the cam lock arrangement.

The second portion may include a pin and the first portion may include a cam that is cam locked with the pin in cam locking together the first and second portions of the cam lock arrangement. Alternatively, the first portion may include a pin and the second portion may include a cam that is cam locked with the pin in the course of cam locking together the first and second portions of the cam lock arrangement. The pin may include a pair of spaced-apart pins, and the cam may include a cam that is cam locked with the pair of spaced-apart pins in the course of cam locking together the first and second portions of the cam lock arrangement.

The cam may be rotated to become cam locked with the pin. Also, the cam may be cam locked with the pin in the course of cam locking together the first and second portions of the cam lock arrangement followed by welding together the pin and cam. The pin may be defined in a material forming the housing. Alternatively, the pin may be welded to the housing prior to the housing having been hermetically sealed or certified.

The pin may include three pairs of spaced-apart pins, and the cam may include three cams that are spaced-apart from each other, each cam being cam locked with a respective one of the three pairs of spaced-apart pins in the course of cam locking together the first and second portions of the cam lock arrangement.

Cam locking together the first and second portions of the cam lock arrangement may compress together the header connector assembly and the housing.

Another implementation of the present disclosure may take the form of a method of manufacturing an implantable pulse generator for administering electrotherapy via an implantable lead The method includes: abutting together a housing and a pre-manufactured header connector assembly, the housing comprising a weld anchor, and the pre-manufactured header connector assembly comprising a connector assembly, a header enclosing the connector assembly, and a weld insert imbedded in a material of the header; and welding together the weld anchor and the weld insert.

The method may further include applying a compressive pre-load to the housing and pre-manufactured header connector assembly in abutting together the housing and pre-manufactured header connector assembly, the compressive pre-load being in existence prior to and during welding together the anchor and the weld insert. The weld insert may include three weld inserts spaced-apart from each other, and the weld anchor may include two pairs of spaced-apart weld anchors. Each pair of spaced-apart weld anchors are paired with two of the weld inserts, and the third weld insert is welded to an RF anchor of the connector assembly. The weld anchor may be defined in a material forming the housing. Alternatively, the weld anchor may be welded to the housing prior to the housing having been hermetically sealed or certified.

While multiple embodiments are disclosed, still other embodiments of the present disclosure will become apparent to those skilled in the art from the following detailed description, which shows and describes illustrative embodiments of the disclosure. As will be realized, the invention is capable of modifications in various aspects, all without departing from the spirit and scope of the present disclosure. Accordingly, the drawings and detailed description are to be regarded as illustrative in nature and not restrictive.

### BRIEF DESCRIPTION OF THE DRAWINGS

FIG. 1 is an isometric view of a proximal end portion (i.e., lead connector end) of a conventional transvenous bipolar pacing lead.

FIG. 2 is an isometric view of a cardiac pacemaker/defibrillator unit (i.e., implantable pulse generator (IPG)) incorporating connector junctions or terminals for communication with one or more electrodes.

FIG. 3A is an isometric view of a representative header.

FIG. 3B is an isometric view of a representative connector assembly used with the header of FIG. 3A to form a header connector assembly.

FIG. 4A is an isometric view of an IPG employing a first anchoring system for anchoring the header connector assembly to a housing to form the IPG.

FIG. 4B is an exploded isometric view of the same IPG wherein the header connector assembly is spaced apart from the housing.

FIG. 5A is a side isometric view of the header connector assembly coupled to the housing and illustrating the first anchoring system wherein the connector pins are received in the cam connectors and the cam connectors are in an unlocked state relative to the connector pins.

FIG. 5B is the same view as FIG. 5A, except the cam connectors are hidden in FIG. 5B to more clearly show the connector pins projecting into the confines of the header connector assembly.

FIG. 6 is a longitudinal side view of an embodiment of a connector pin.

FIG. 7 is an isometric view of a cam connector that is configured for cam lock engagement with the head feature of one or more connector pins configured as depicted in FIG. 6.

FIG. 8A is an isometric view of the connector pins received in the respective transverse pin-receiving slots of the cam connector and illustrating an unlocked arrangement between the cam connector and the connector pins.

FIG. 8B is an isometric view similar to FIG. 8A, except the cam connector is rotating from the unlocked arrangement towards a locked arrangement.

FIG. 8C is an isometric view similar to FIG. 8B, except from another perspective and showing the locked arrangement between the cam connector and the connector pins.

FIG. 8D is a side isometric view of the cam connector in the locked arrangement.

FIG. 9A is another isometric view of the cam connector and connector pins in an unlocked arrangement.

FIG. 9B is an isometric view similar to FIG. 9A, except of the cam connector and connector pins in the locked arrangement.

FIG. 9C is a transverse cross section through the cam connector and one of the connector pins as taken along section line 9C-9C of FIG. 9B.

FIG. 10 is a transverse cross sectional isometric view along the longitudinal axis of one of the three cam connectors as taken along section line 10-10 of FIG. 4A.

FIG. 11 is a transverse cross sectional isometric view along the longitudinal axis of another of the three cam connectors as taken along section line 11-11 of FIG. 4A.

FIG. 12 is a transverse cross sectional isometric view along the longitudinal axis of yet another of the three cam connectors as taken along section line 12-12 of FIG. 4A.

FIG. 13 is a force diagram of generally the same view as depicted in FIG. 10.

FIG. 14 is a another force diagram depicted on a transverse cross section of the header connector assembly and the housing, the cross section taken along the longitudinal axis of a cam connector.

FIG. 15 is a flow chart illustrating a method of manufacturing the IPG of FIGS. 4A and 4B.

FIG. 16 is an isometric view of an IPG wherein its two modules, namely, the header connector assembly and the housing, are anchored together via a welded arrangement.

FIG. 17 is an isometric view of a pre-molded header connector assembly with three weld inserts molded into the header of the pre-molded connector assembly.

FIG. 18A is an isometric view of a first type of weld insert.

FIGS. 18B and 18C are opposite isometric views of another type of weld insert.

FIG. 19 is an isometric view of a weld anchor.

FIG. 20 is an isometric view of the IPG generally opposite from the view depicted in FIG. 16.

FIG. 21 is an isometric transverse cross sectional elevation of the IPG as taken along section line 21-21 of FIG. 16.

FIG. 22 is an isometric transverse cross sectional elevation of the IPG as taken along section line 22-22 of FIG. 16.

FIG. 23 is an enlarged isometric transverse cross sectional view of a welded arrangement depicted in FIG. 21.

FIG. 24 is a transverse cross section elevation of the IPG similar to that depicted in FIG. 21.

### DETAILED DESCRIPTION

Implementations of the present disclosure involve an implantable pulse generator (IPG) for administering electrotherapy or other neurostimulation via an implantable lead having a lead connector end on a proximal end of the implantable lead. The IPG includes a housing or can and a connector assembly enclosed in a header, both of which are coupled to the housing or can. The header and connector assembly combine to form at least one lead connector receiving bore or receptacle that includes electrical contacts that make electrical contact with corresponding electrical terminals on the lead connector end on the proximal end of the implantable lead when the lead connector end is plugged into or otherwise received in the lead connector receiving bore or receptacle. Via the electrical connection between the corresponding electrical terminals of the lead connector end and the electrical contacts of the lead connector receiving bore, electrical signals can be administered from the IPG and through the lead to patient tissue. Similarly, but in reverse, electrical signals originating in patient tissue can travel via the lead to the IPG to be sensed at the IPG.

The IPG configurations and methods of assembly disclosed herein are advantageous for at least the reason that they eliminate the backfill process. Specifically, the IPGs disclosed herein are configured such that a pre-molded header and connector assembly enclosed therein are anchored to the housing via a mechanical cam arrangement or a welded arrangement, thereby facilitating assembly methodologies that eliminate the traditional backfill process. In some embodiments, the pre-molded header is a result of pre-casting process or a pre-injection process.

The IPG configurations and methods of assembly disclosed herein provide substantial cost and time savings over those IPG configurations and methods of assembly that are associated with the traditional backfill process. Also, the IPG configurations and methods of assembly disclosed herein result in a secure attachment between the housing and the header and connector assembly.

Before beginning a detailed discussion of the assembly of the header and the connector assembly enclosed therein onto the housing, a general discussion is first given regarding features of a common lead connector end at the proximal end of an implantable medical lead followed by a general discussion of the features of an IPG.

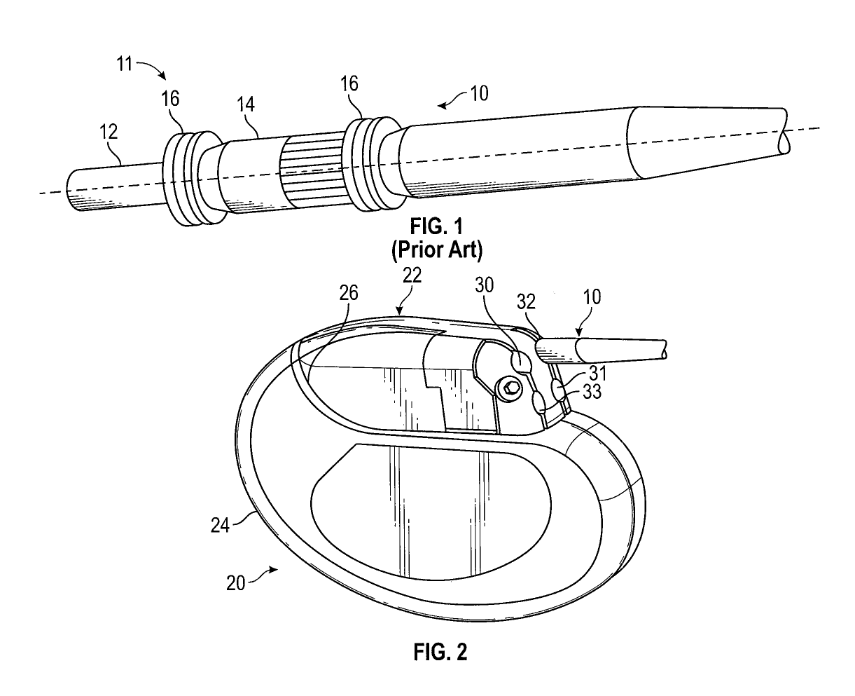

FIG. 1 shows a proximal end portion 10 of a conventional transvenous, bipolar pacing lead, but is generally representative of any type of implantable lead whether in the cardiac, pain management or other medical treatment space. The diameter of such a lead may be made a sufficiently small diameter to facilitate the lead's implantation into small veins such as those found in the coronary sinus region of the heart and to allow implantation of a plurality of leads into a single vessel for multi-site or multi-chamber pacing. It should be understood, however, that other lead designs may be used, for example, multipolar leads have proximal ends portions that are bifurcated, trifurcated or have other branched configurations. While the lead whose proximal end is shown in FIG. 1 is of the bipolar variety, there are unipolar leads that carry but a single electrode, and multipolar leads that have more than two electrodes.

As is well known in the art, bipolar coaxial leads typically consists of a tubular housing of a biocompatible, biostable insulating material containing an inner multifilar conductor coil that is surrounded by an inner insulating tube. The inner conductor coil is connected to a tip electrode on the distal end of the lead. The inner insulating tube is surrounded by a separate, outer multifilar conductor coil that is also enclosed within the tubular housing. The outer conductor coil is connected to an anodal ring electrode along the distal end portion of the lead. The inner insulation is intended to electrically isolate the two conductor coils preventing any internal electrical short circuit, while the housing protects the entire lead from the intrusion of body fluids. These insulating materials are typically either silicone rubber or polyurethane. More recently, there have been introduced bipolar leads in which multifilar cable conductors contained within multilumen housings are substituted for the conductor coils in order to reduce even further the overall diameter of the lead.

The proximal lead end portion 10 shown in FIG. 1 includes a lead connector end 11 that conforms to the IS-1 standard, comprising a pair of coaxial spaced-apart electrical terminals including a tip terminal 12 and a ring terminal 14. The tip terminal 12 is electrically connected by means of the inner conductor coil to the tip electrode at the distal end of the lead, while the ring terminal 14 is electrically connected to the anodal ring electrode by means of the outer conductor coil. The tip and ring terminals of the lead connector end may each be engaged by a conductive garter spring contact or other resilient electrical contact element in a corresponding lead connector receiving bore of the header, the resilient electrical contact element being carried by a connector assembly enclosed in the header as described below. The lead connector end 11 on the proximal lead end portion 10 further comprises spaced-apart pairs of seal rings 16 for abutting against in a fluid-sealing manner the inner circumferential surface of the lead connector receiving bore of the header, thereby preventing body fluids from reaching the electrical terminals and contacts when the lead connector end 11 is plugged into the corresponding lead connector receiving bore. With the lead connector end 11 of the lead inserted in the lead connector receiving bore of the header and connector assembly, the tip and ring terminals 12 and 14 are electrically coupled via the contacts of the connector assembly and a feedthru to the electronic circuits within the hermetically sealed housing of the IPG (e.g., cardiac pacemaker, ICD, or other implantable tissue stimulation and/or sensing device such as those used in pain management, etc.).

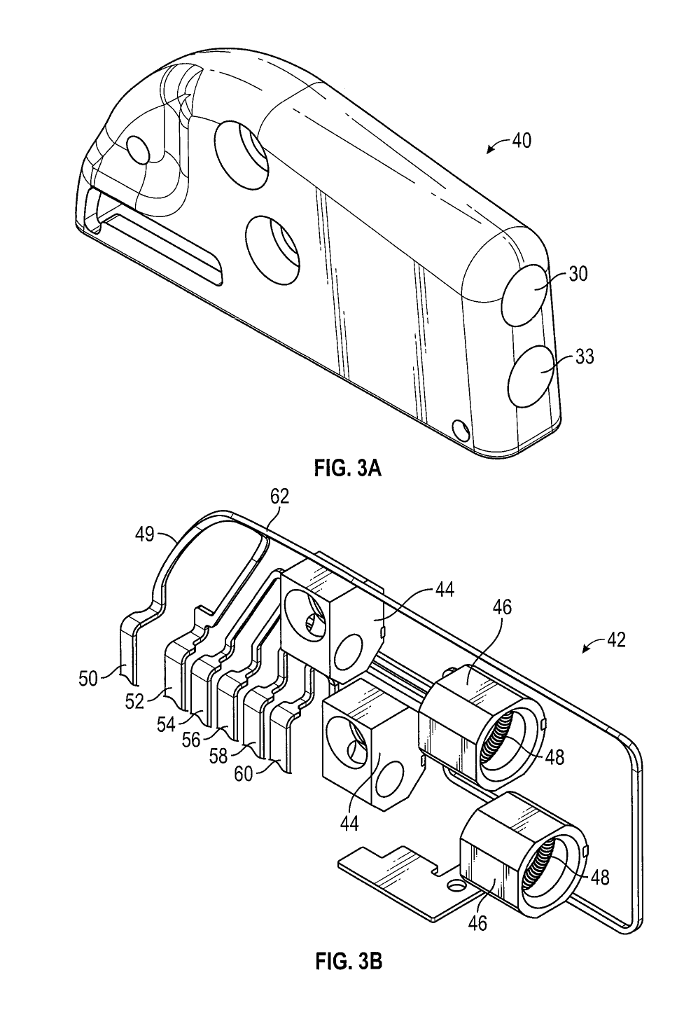

FIG. 2 shows a multi-site or multi-chamber cardiac pacemaker/defibrillator unit that is generally representative of any type of IPG 20 incorporating a header connector assembly 22 coupled to a housing 24. The header connector assembly 22 includes a header 40 enclosing a connector assembly 42, both of which are depicted respectively in FIGS. 3A and 3B discussed below. The IPG 20 is of a conventional design, including a hermetically sealed housing 24, which is also known as a can or casing. The housing 24 encloses the electronic components of the IPG 20 with the header connector assembly 22 mounted along a top edge 26 of the housing 24.

FIG. 2 illustrates that, in some embodiments, the header connector assembly 22 may include four or more lead connector receiving bores or receptacles 30, 31, 32 and 33 for receiving the lead connector ends of four implantable leads. FIG. 2 also shows the proximal end portion 10 of a lead, wherein the lead connector end on the proximal end portion 10 of the lead is received in a corresponding receptacle 32. In other embodiments, the header connector assembly 22 includes two receptacles comprising a single pair of receptacles (i.e., receptacles 30 and 33) for receiving the proximal ends of leads such as, for example, conventional bipolar leads and/or conventional cardioverting and/or defibrillating leads.

FIG. 3A is an isometric view of a representative header 40 and FIG. 3B is an isometric view of a representative connector assembly 42. Unlike the header connector assembly 22 of FIG. 2, the header 40 of FIG. 3A only has a single pair of receptacles 30 and 33. However, in other embodiments, the header 40 of FIG. 3A may have two or more pairs of receptacles similar to the embodiment of FIG. 2.

As illustrated in FIG. 3B, the connector assembly 42 includes tip blocks 44 and ring blocks 46. The ring blocks 46 include spring contacts 48. Each electrical block 44 and 46 of the connector assembly 42 serves as an electrical contact of the connector assembly 42. Thus, as can be understood from FIGS. 1, 2 and 3B, each tip block 44 is configured to receive and make electrical contact with the tip terminal 12 of a lead connector end 11 received in the corresponding receptacle 30, 33 of the header 40. Similarly, each ring block 46 is configured to receive and make electrical contact with the ring terminal 14 of a lead connector end 11 received in the corresponding receptacle 30, 33 of the header 40. While the connector assembly 42 of FIG. 3B is of an IS-1 configuration, other configurations (e.g., IS-4, etc.) are used in other embodiments. While the connector assembly 42 of FIG. 3B only depicts two pairs of blocks 44, 46, in other embodiments where the header includes more than a single pair of receptacles 30, 33 (e.g., two pairs of receptacles 30, 31, 32, 33 as indicated in FIG. 2), the connector assembly 42 will have a four pairs of blocks 44, 46.

As shown in FIG. 3B, the connector assembly 42 also includes an antenna 49, a an RF anchor tab 50, an RF pin tab 52, an A-tip tab 54, an A-ring tab 56, an RV-ring tab 58, an RV-tip tab 60, and a ribbon carrier 62 and other conductors that extend between the various tabs and their respective electrical contacts of the connector assembly or other components thereof. The various tabs are welded to corresponding terminals extending from circuitry of the IPG 20 contained in the housing 24 of the IPG 20 depicted in FIG. 2 when the header connector assembly 22 is joined with the housing 24 to form the IPG 20. The connector assembly 42 is manufactured of materials and via methods known in the industry. The connector assembly 42 is molded into the header 40 to form the header connector assembly 22 of FIG. 2, which can be considered a first module that is then anchored to a second module in the form of the housing 24. The header connector assembly 22 (i.e., first module) is anchored to the housing 24 (i.e., the second module) via any of the systems and methods described below to form the IPG 20.

### A. Anchoring Header Connector Assembly to Housing Via Cam and Pin Arrangement

A first anchoring system and method is used to securely attach together two IPG modules, namely, the header connector assembly 22 and the housing 24, to form the IPG 20. The anchoring system and method function through the interaction of two components in the form of connector pins 66 and cam connectors 64. The connector pins of one IPG module is inserted into cam connectors of another IPG. Turning each cam connector about its respective connector pins tightens the attachment of the cam connector to the connector pins, thereby providing compression between two modules. Thus, turning all of the cam connectors to fasten with associated connector pins increases compression and attachment strength, and the anchoring mechanism formed by the cam connectors and connector pins works to lock in place one IPG module relative to another IPG module, in other words, the header connector assembly 22 relative to the housing 24.

Access points 92 in the cam connectors 64 allow welding of the cam connectors to the connector pins 66 and/or adhesive injection into the cam connectors and around the connector pins about which the cam connectors are locked, thereby keeping the attachment between the cam connectors and connector pins secure and sealed. Access points in the header 40 of the header connector assembly 22 allow welding of components of the header to components of the housing 24 and/or adhesive injection into voids between the header connector assembly and the housing to keep the attachment secure and sealed between the header connector assembly and the housing.

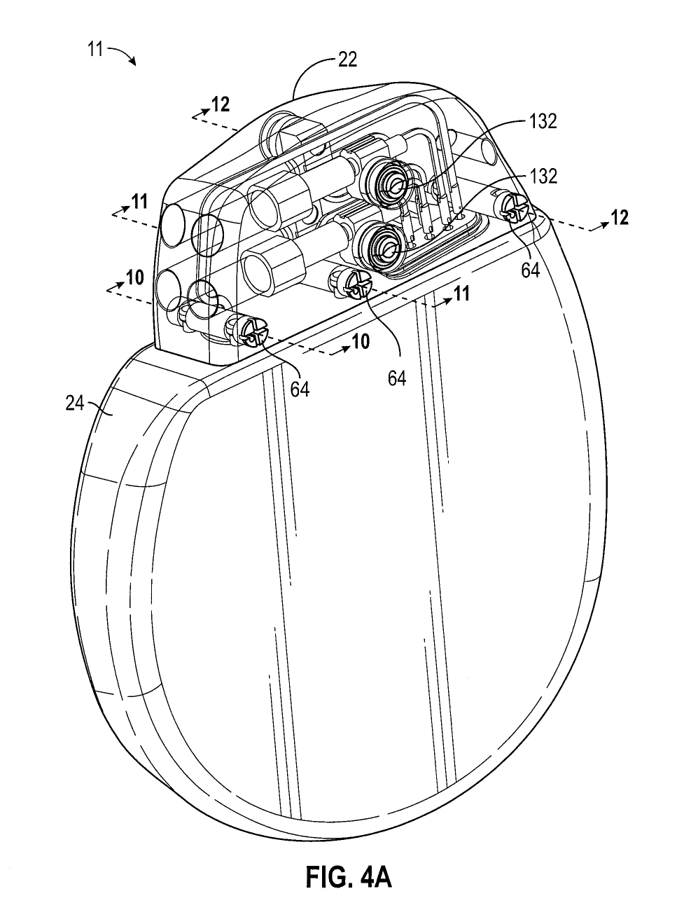

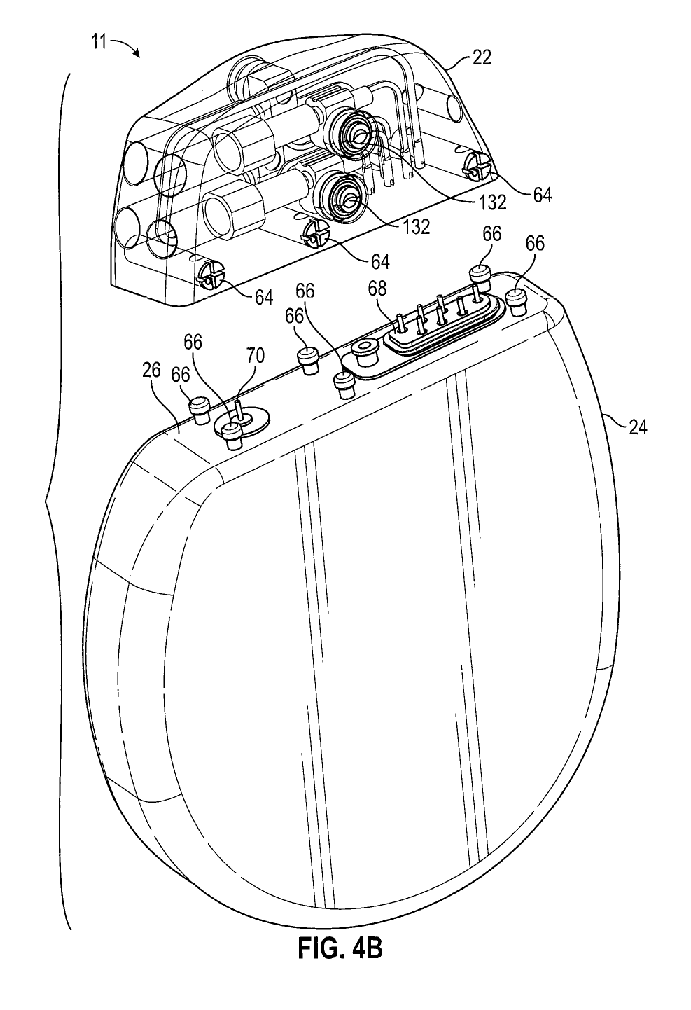

To begin a detailed discussion of the first anchoring system and method, reference is made to FIGS. 4A and 4B, which are, respectively, an isometric view of an IPG 20 employing the first anchoring system and an exploded isometric view of the same IPG 20 wherein the header connector assembly 22 is spaced apart from the housing 24. The header connector assembly 22 includes three cam connectors 64 spaced apart from each other along the base of the header connector assembly. The housing 24 includes a top surface 65 and three pairs of connector pins 66 similarly spaced apart from each other along the top surface 65 of the housing, each pin 66 also laterally spaced apart from its sibling pin 66 that makes up that specific pair of connector pins 66. While the cam connectors 64 are part of the header connector assembly 22 and the connector pins 66 are part of the housing 24 in the embodiment depicted in FIGS. 4A and 4B, in some embodiments, the connector pins 66 are part of the header connector assembly 22 and the cam connectors 64 are part of the of the housing 24.

As depicted in FIG. 4B, a feedthru 68 and an antenna 70 with their associated terminals extend outwardly from the top surface 26 of the housing from their respective connections to the electronic circuitry contained within the confines of the housing 24. Subsequent to the header connector assembly 22 being anchored to the housing 24 via the cam and pin anchoring system, the wires of the feedthru 68 and antenna 70 are welded to their respective tabs 50, 52, 54, 56, 58, and 60 discussed above with respect to FIG. 3B.

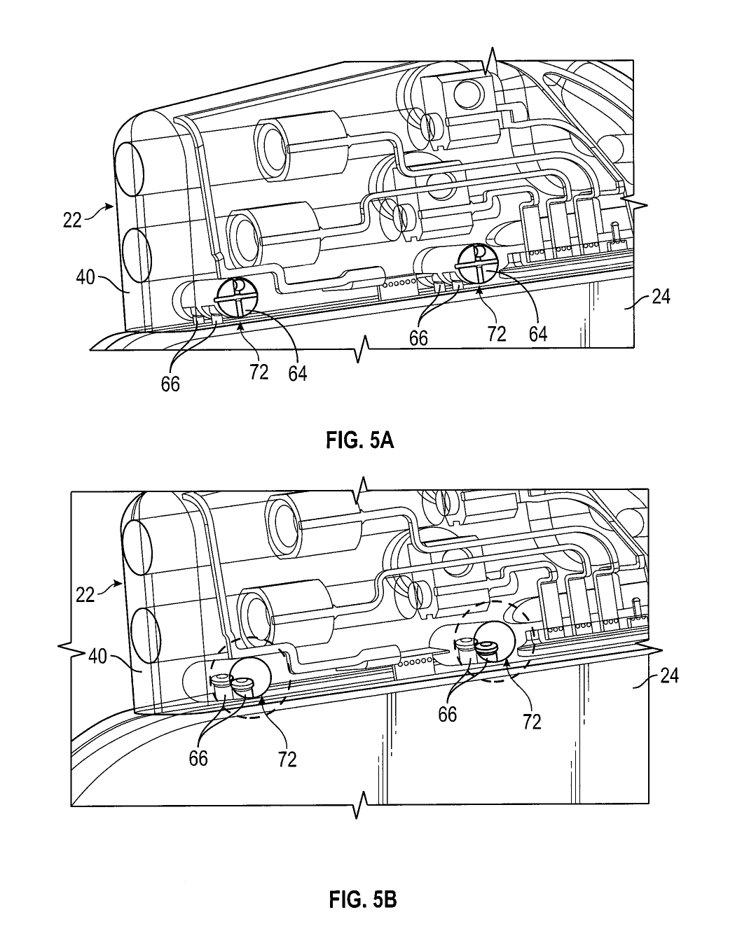

FIGS. 5A and 5B are, respectively, the same side isometric views of the header connector assembly 22 abutting the housing 34 in preparation for the cam connectors 64 to be rotated from an unlocked state (shown) to a locked state about the pin connectors 66, the difference between FIGS. 5A and 5B being that the cam connectors 64 are hidden in FIG. 5B to more clearly show the connector pins 66 projecting into the confines of the header connector assembly 22. As can be understood from FIGS. 5A and 5B, the cam connectors 64 are pivotally supported in cylindrical laterally extending holes 72 in the header 40 of the header connector assembly 22. In one embodiment, the longitudinal axis of each cam connector and its respective hole 72 is parallel or at least substantially parallel to the top surface 26 (shown in FIG. 4B) of the housing 24.

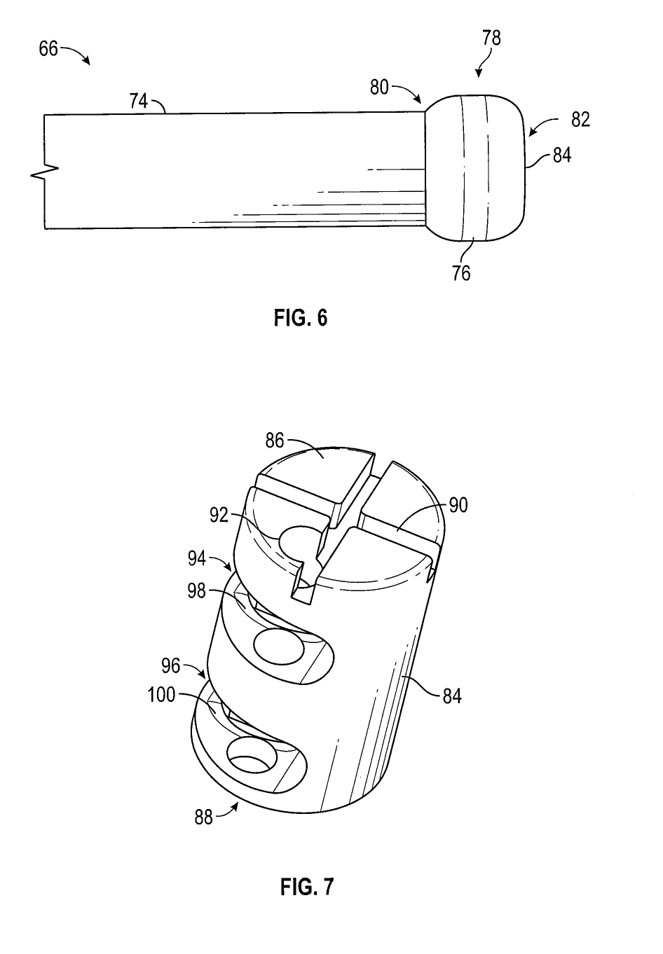

As indicated in FIG. 6, which is a longitudinal side view of an embodiment of a connector pin 66, the connector pin has a proximal shaft portion 74 that extends from its respective IPG module to distally terminate in a head feature 76 having a transverse diameter or width that is larger than the diameter of the shaft portion 74. The head feature 76 may be generally rounded on its sides 78 and be generally truncated or flat on its proximal end 80 and distal end 82 such that its proximal end transitions abruptly from an arcuate shape to the shaft portion 74 and its distal end transitions abruptly from an arcuate shape to a flat distal face 84 normal to the longitudinal axis of the shaft portion 74. While the connector pin 66 depicted in FIG. 6 illustrates a cylindrical shaft portion 74 with a truncated rounded head feature 76, the shaft portion 74 and head feature 76 may have other shapes and configurations so long as they can achieve a cam lock interface with a complementary cam connector 64. Also, the connector pin 66 may be any other type of connector member 66, whether of a type that projects outwardly similar to the projecting connector pin depicted in FIG. 6, a recessed configuration, a hook-type configuration or any other configuration that can achieve a cam lock interface with a complementary cam connector 64. The connector pins 66 may be formed or machined from a variety of materials including, for example, stainless steel, titanium, titanium alloy, 17-4 PH, MP35N or etc.

FIG. 7 is an isometric view of a cam connector 64 configured for cam locking engagement with the head feature 76 of one or more connector pins 66 configured as depicted in FIG. 6. The cam connector 64 is generally cylindrical such that it can be rotated about its longitudinal axis within the confines of the hole 72 in the header 40 to bring the cam connector into cam locked engagement with the head feature 76 of one or more connector pins 66. The cam connector includes a cylindrical outer surface 84, opposed ends 86 and 88, a tool engagement feature 90, an access bore 92, and transverse pin-receiving slots 94 and 96. The cylindrical outer surface extends between the opposed ends. A tool engagement feature 90 is defined in one or both opposed ends 86 and 88 and may be in the form of a screwdriver tip interface, an Allen wrench interface, a bolt nut interface, etc. The access bore 92 is radially offset from the longitudinal center axis of the cam connector and extends lengthwise through the cam connector parallel to the longitudinal center axis. Transverse pin-receiving slots 94 and 96 extend radially inward from the cylindrical outer surface 84 and each such slot includes a respective cam surface 98 and 100 that cams against the head feature 76 of respective connector pins 66 when the head features are received in the respective slots and the cam connector is rotated appropriately. The cam connector 64 may be formed or machined from a variety of materials including, for example, stainless steel, titanium, titanium alloy, 17-4 PH, MP35N or etc.

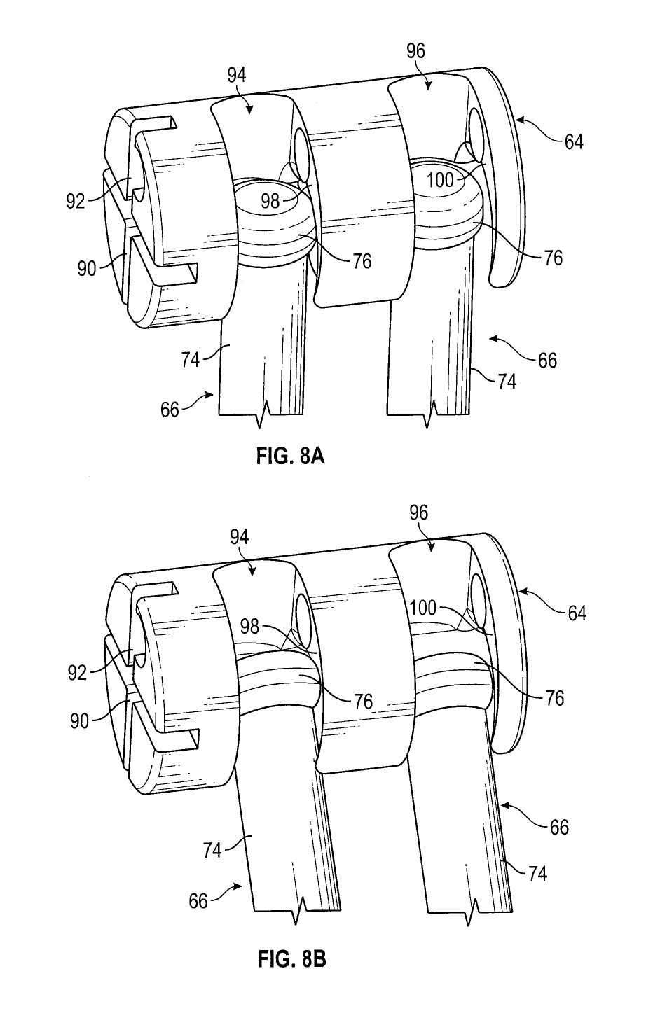

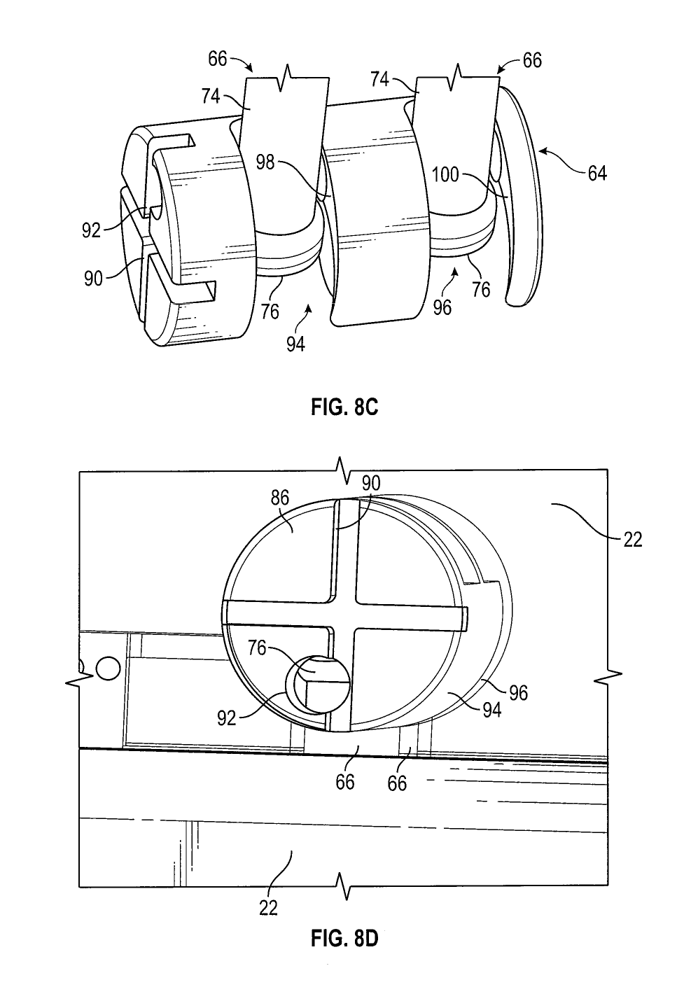

FIGS. 8A, 8B and 8C are isometric views of the connector pins 66 received in the respective transverse pin-receiving slots 94, 96 of the cam connector 64, wherein the figures respectively show an unlocked arrangement, the cam connector beginning to rotate from the unlocked arrangement towards a locked arrangement, and the locked arrangement. As can be understood from FIG. 8A, when the cam connector 64 is rotated relative to the connector pins 66 so as to be in an unlocked relationship with the connector pins although the connector pins are received in the transverse pin-receiving slots 94, 96 of the cam connector, the cam surfaces 98, 100 do not engage the head feature 76 of the respective connector pins.

As can be understood from FIGS. 8A-8C, as the cam connector 64 is increasingly rotated from the unlocked arrangement of FIG. 8A to the locked arrangement of FIG. 8C, the cam surfaces 98, 100 increasingly engage the head feature 76 of the respective connector pins 66, placing the connector pins in substantial tension. The tensioning of the connector pins results in the IPG modules 22 and 24 being compressed against each other, as can be understood from 4A-5B and as discussed in greater detail below. Advantageously and as can be understood from FIGS. 4A-8C, since the anchoring mechanism functions by the cam connectors 64 rotating in place relative to their respective connector pins 66, no additional space is required when going between the loose or unlocked state and the cam locked or anchored state, which is advantageous where space is extremely limited as in the context of IPGs.

As can be understood from FIG. 8D, which is a side isometric view of the cam connector 66 in cam locked engagement with its two respective connector pins 66, the access bore 92 can be used for multiple purposes. For example, the access bore 92 can provide access for welding the cam connector to the respective connector pins, thereby permanently fixing the cam connector and connector pins in the cam locked engagement. Alternatively or additionally, the access bore 92 can provide access for adhesive injection into the voids of the cam connector and any surrounding voids between anchored together IPG modules 22, 24. In addition to the adhesive filling said voids, the adhesive also permanently fixes the cam connector and connector pins in the cam locked engagement.

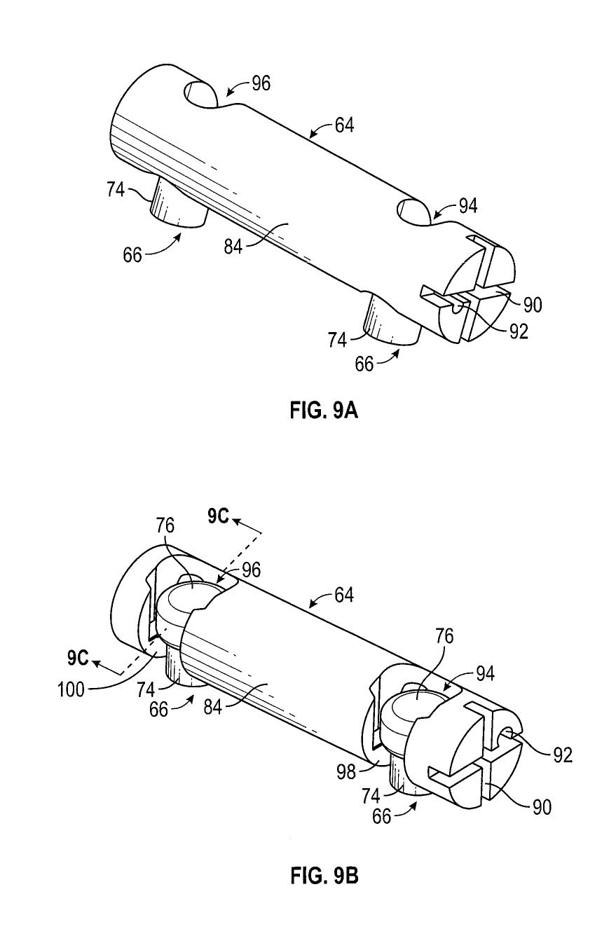

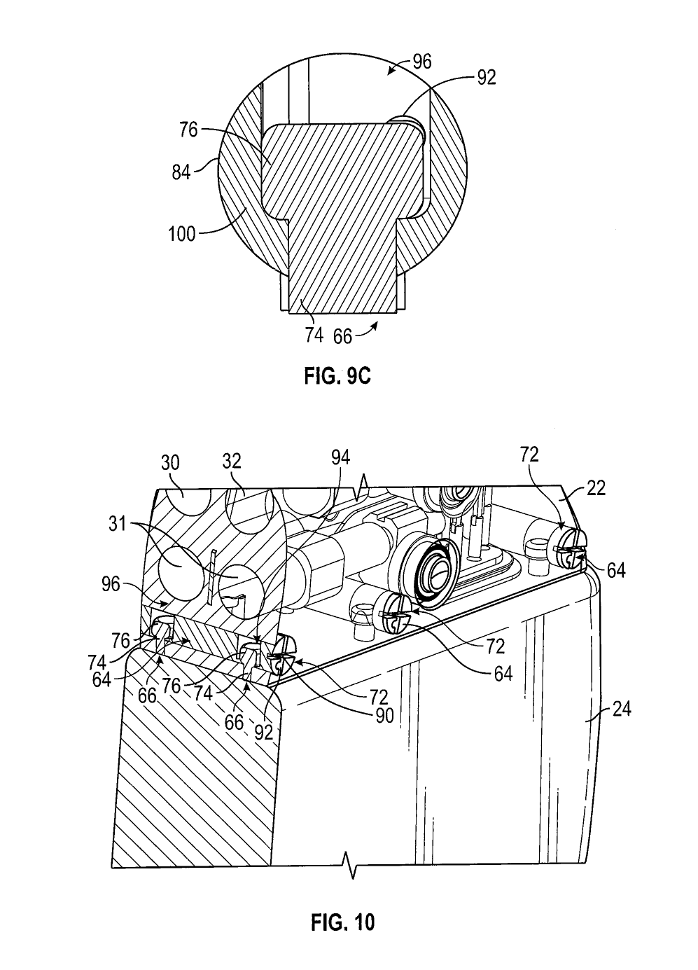

FIGS. 9A and 9B are isometric views of the cam connector 64 and connector pins 66 in an unlocked arrangement and a locked arrangement, respectively. FIG. 9C is a transverse cross section through the cam connector and one of the connector pins as taken along section line 9C-9C of FIG. 9B. Many of the teachings discussed with respect to FIGS. 8A-8D can be further understood from FIGS. 9A-9C.

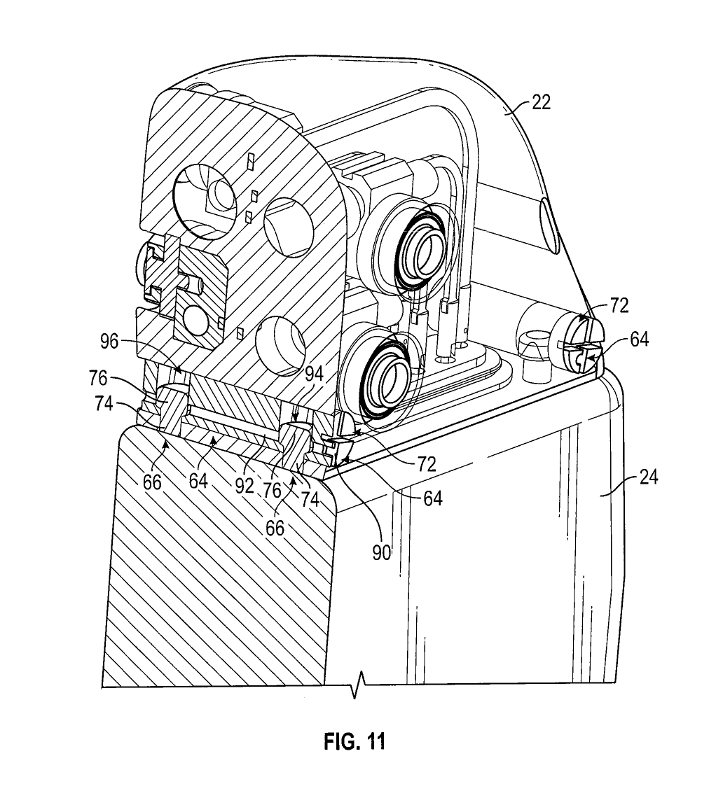

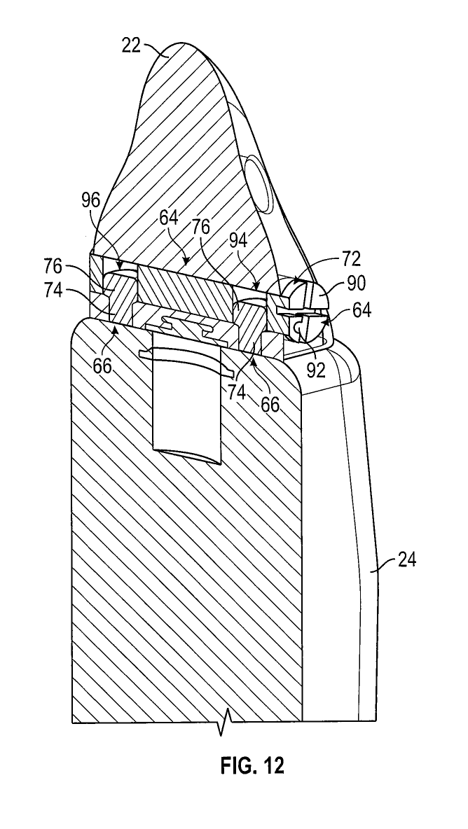

FIGS. 10-12 are each transverse cross sectional isometric views aligned with the respective longitudinal axes of the three cam connectors 64 as taken along respective section lines 10-10, 11-11 and 12-12 of FIG. 4A. As can be understood from FIG. 10, in one embodiment, three cam connectors 64 extend transversely across the header connector assembly 22 and are generally evenly spaced apart from each other along the length of the header connector assembly. In other embodiments, there may be a greater or lesser number of cam connectors 64 supported in the header connector assembly 22.

As can be understood from FIGS. 10-12, in one embodiment, each cam connector 66 engages a pair of spaced-apart connector pins 66 that extend upward from the top surface 26 of the housing 24, as also indicated in FIG. 4B. The pairs of connector pins are spaced-apart along the length of the top surface of the housing in a spaced arrangement that matches spacing of the cam connectors 64 along the length of the header connector assembly. As best illustrated in FIG. 4B and can also be understood from FIGS. 10-12, each connector pin 66 of a pair of connector pins is located near a lengthwise side edge of the top surface 26 of the housing 24 such that the connector pins of each pair of connector pins are transversely spaced apart from each other as greatly as possible, generally speaking. Such a transverse spacing of the connector pins 66 matches the spacing of the pin-receiving slots 94, 96 along the length of each respective cam connector 64, as illustrated in FIGS. 10-12.

As shown in FIGS. 10-12, the tool engagement feature 90 of each cam connector 64 is exposed in, or even projects from, the side of the header connector assembly 22. As a result, a screw driver, Allen wrench or other appropriate tool can engage the tool engagement feature 90 to cause the cam connector 64 to rotate about its longitudinal axis and within the header connector assembly 22 to cause the cam surfaces of the pin-receiving slots 94, 96 to cam lock about the head features 76 of the respective connector pins 66.

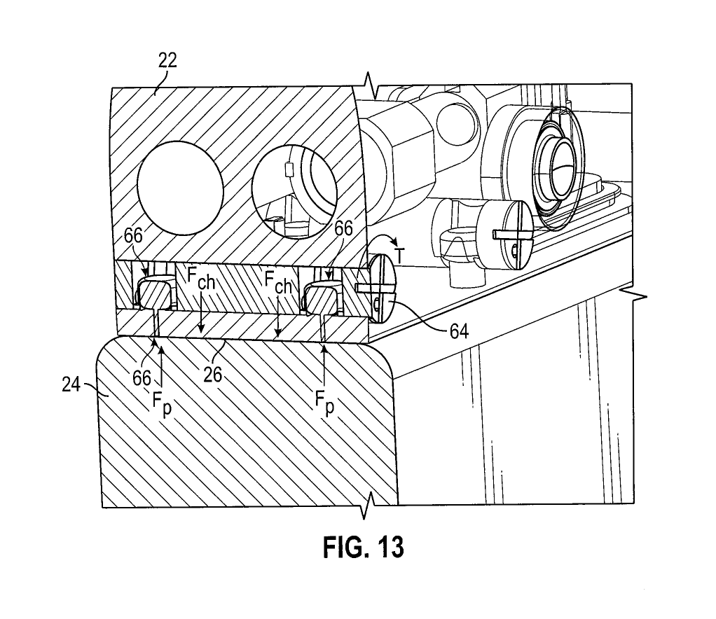

FIG. 13 is a force diagram of generally the same view as depicted in FIG. 10, wherein the header connector assembly is pre-loaded in compression against the housing. As shown in FIG. 13, torquing of the cam lock mechanism creates a compression force between the header connector assembly 22 and the housing 24, wherein T equals the tightening torque on the cam lock mechanism, Fₚ equals the pulling up force on the pin, and F_{ch} equals the pushing down force of the header connector assembly onto the housing. The pushing down force F_{ch} can be substantial enough to create a seal between the bottom peripheral edge of the header connector assembly 22 against the top surface 26 of the housing 24. This seal can prevent any sealant injected into the header connector assembly 22 from leaking from between the interface surface between the bottom peripheral edge of the header connector assembly 22 and the top surface 26 of the housing 24. Further, the pre-loaded nature of the resulting anchoring of the header connector assembly to the housing results in an IPG 20 that is more capable of withstanding cyclic loading without fatigue failure.

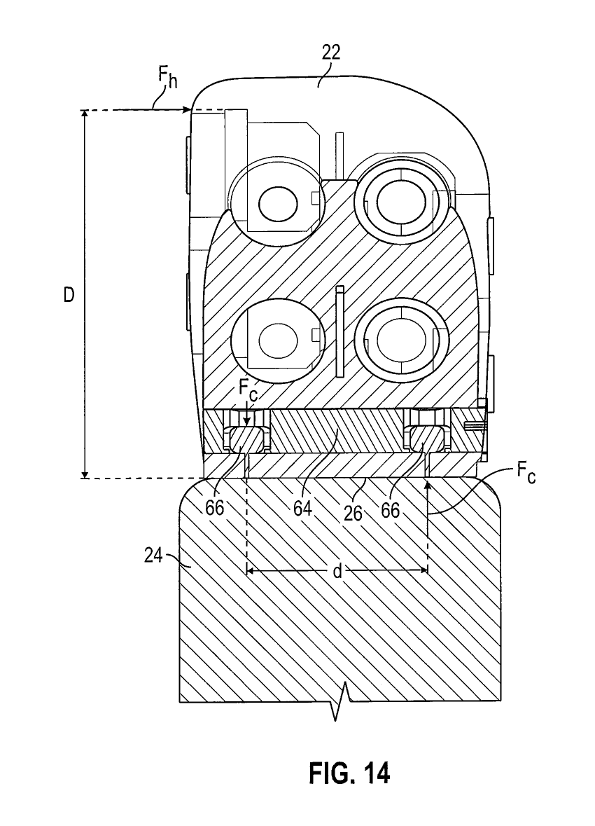

FIG. 14 is a another force diagram depicted on a transverse cross section of the header connector assembly 22 and the housing 24, the cross section taken along the longitudinal axis of a cam connector 64. As can be understood from FIG. 14, Fₕ equals a force exerted on a top edge of the header connector assembly and extending transverse to the header connector assembly. F_{c} equals the resultant reaction force on the cam lock mechanism when D equals a torque arm extending between Fₕ and the cam lock mechanism and d equals another torque arm extending between the connector pins. Since the summation of torques must be equal (Fₕ×D=F_{c}×d×3), then F_{c}=Fₕ×D/d×3. Thus, the larger the distance (d) between the connector pins 66, the less force on the connector pins resulting from a transverse force (Fh) exerted on the header connector assembly 22. The less force on the connector pins, the less stress on the header connector assembly and the less chance of a related structural failure, including adhesive separation or delamination.

As can be understood from FIG. 14, employing an anchoring system employing cam connectors 64 cam locking with pairs of connector pins 66 that are substantially transversely spaced-apart as far as practical secures the entire width of the header connector assembly and distributes stress from a bending moment that might be applied to the header during the life of the IPG 20. Also, as can be understood from FIG. 13, because the connector pins 66 are both transversely and longitudinally spaced apart on the top surface 26 of the housing 24 as generally far as possible and generally evenly, the compression force between the housing and the header connector assembly is generally evenly distributed along the periphery of the interfacing between the housing and the header connector assembly, thereby creating a continuous and substantial compression seal between the two IPG models along the periphery boundary of the interface between the two IPG models. Finally, as can be understood from FIGS. 13 and 14, the cam connectors 64 cam locked with their respective pairs of connector pins 66 secures the pre-molded header connector assembly 22 to the housing 24 in the x, y, z, and τ (rotation) axes.

While the above-discussed figures depict an embodiment employing a pair of connector pins 66 for each cam connector 64, in some embodiments where the IPG 20 is of a smaller width, each cam connector 64 will only interface with a single connector pin. In other embodiments, where the IPG 20 has a wider width, each cam connector 64 may interface with three or more connector pins 66. Also, while the above-discussed figures depict an embodiment employing three cam connectors 64, in some embodiments where the IPG 20 is longer or shorter, there will be more than three cam connectors or less than three cam connectors, respectively.

In one embodiment, the connector pins are formed, machined or otherwise defined in the material forming the housing, said material being titanium, titanium alloy, MP35N, stainless steel, or etc. In another embodiment, the connector pins are welded or otherwise affixed to the housing prior to the housing having been hermetically sealed or achieving it hermetic certification.

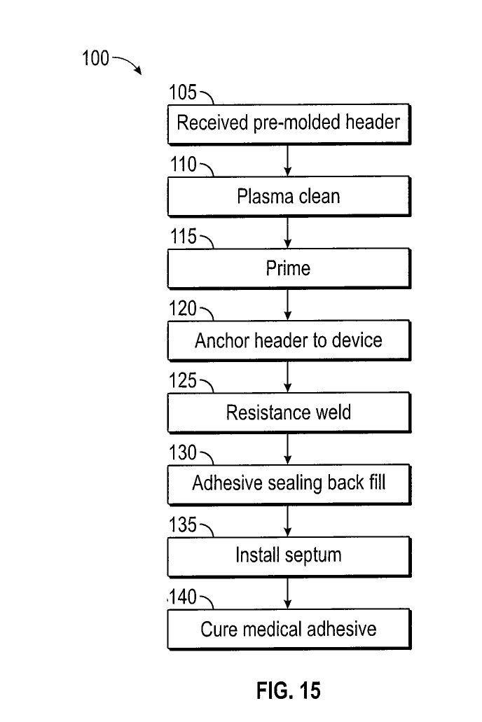

As can be understood from FIG. 15, which is a flow chart illustrating a method 100 of manufacturing the IPG 20 of FIGS. 4A and 4B, a pre-molded header connector assembly 22 is received [block 105]. The pre-molded header connector assembly 22 can be an epoxy casted header connector assembly 22 or an injected polymer header connector assembly 22. In one embodiment, the epoxy of the epoxy casted header connector assembly includes, epoxy or etc. In one embodiment, the polymer of the injected polymer header connector assembly includes polyurethane, tecothane, pellethane, bionate, or etc.

The pre-molded header connector assembly 22 is plasma cleaned [block 110], primed [115] and then anchored to the housing 24 via the above-described anchoring system that employs the cam connectors 64 and the pairs of connector pins 66 [block 120]. The cam connectors are resistance welded to their respective connector pins, as are the electrical connections between the various tabs 50, 52, 54, 56, 58, 60 of the connector assembly 42 (see FIG. 3B) and the various wires of the feedthru 68 and antenna 70 (see FIG. 4B) [block 125]. Adhesive is injected into the voids of the header connector assembly 22 and the voids between the header connector assembly and the housing 24 [block 130]. This adhesive backfilling process may employ an adhesive such as, for example, a silicone adhesive, or etc. Septums 132 (shown in FIGS. 4A and 4B) are installed over the setscrews used to secure lead connector ends in the conductor blocks 44 (see FIG. 3B) of the connector assembly 42. The adhesive is then cured [block 140].

Unlike the above-described system and methods, current attachment methods must employ a backfill process to structurally join the header connector assembly to the housing because the housing is formed of titanium and epoxy can join the header connector assembly to such a housing material. Advantageously allowing for the elimination of such epoxy backfilling processes for the purpose of structurally joining the header connector assembly and the housing, the above-described system and method employs an anchoring mechanism in the form of cam connectors and connector pins that is independent of the material of the header connector assembly 22 and the housing 24. As a result, the above-described system and method allows any biocompatible material to be used for the backfill process described herein, which is more on the order of a simple sealing process and not focused on structural attachment between the housing and the header connector assembly, the structural attachment being addresses by the anchoring mechanism formed by the cam connectors and the connector pins.

### B. Anchoring Header Connector Assembly to Housing Via Welding

A second anchoring system and method is used to securely attach together two IPG modules, namely, the header connector assembly 22 and the housing 24, to form the IPG 20. The anchoring system and method include a welded arrangement 200 between the header connector assembly and the housing. The header connector assembly is in the form of a pre-molded assembly including one or more weld inserts 202. The pre-molded header connector assembly is clamped against the housing and the weld inserts 202 are welded to weld anchors 204 located on the housing top surface 26, after which any backfilling and further processing of the resulting IPG can be undertaken. Thus, welded arrangements 200 between the two IPG modules work to lock in place one IPG module relative to another IPG module, in other words, the header connector assembly 22 relative to the housing 24.

Access windows 206 in the header 40 of the header connector assembly 22 allow welding access to the weld inserts 202 and the corresponding weld anchors 204. These windows 206 also allow access for adhesive injection into the header connector assembly 22 to further secure the header connector assembly to the housing and seal any openings in the header connector assembly and between the housing and the header connector assembly. Other access points in the header 40 of the header connector assembly 22 allow welding of components of the connector assembly 42 to components of the housing 24 and/or adhesive injection into voids between the header connector assembly and the housing to keep the attachment secure and sealed between the header connector assembly and the housing.

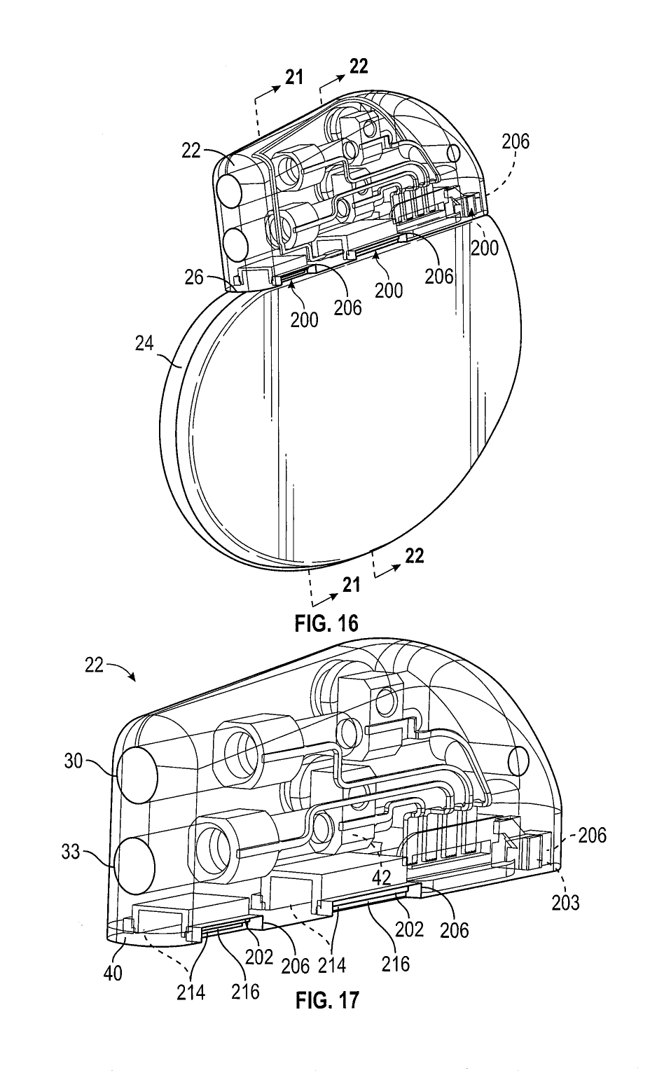

To begin a detailed discussion of the second anchoring system and method, reference is made to FIG. 16, which is an isometric view of an IPG 20 wherein its two modules, namely, the header connector assembly 22 and the housing 24, are anchored together via welded arrangements 200. As shown in FIG. 16, in one embodiment, the IPG 20 employs three welded arrangements 200 that are spaced apart from each other along the length of the top surface 26 of the housing 24. Weld access windows 206 are immediately adjacent each side of each welded arrangement 200.

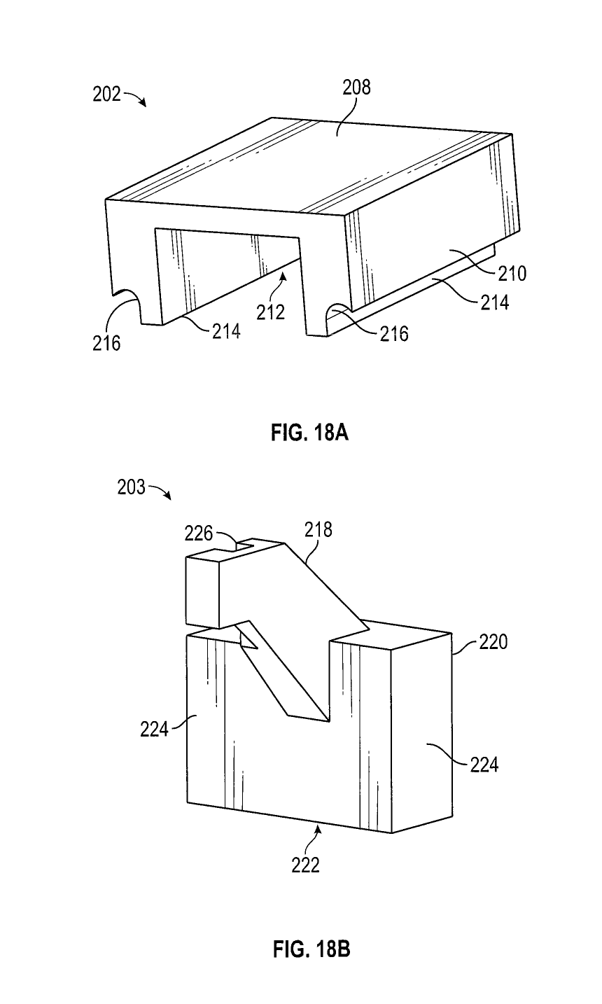

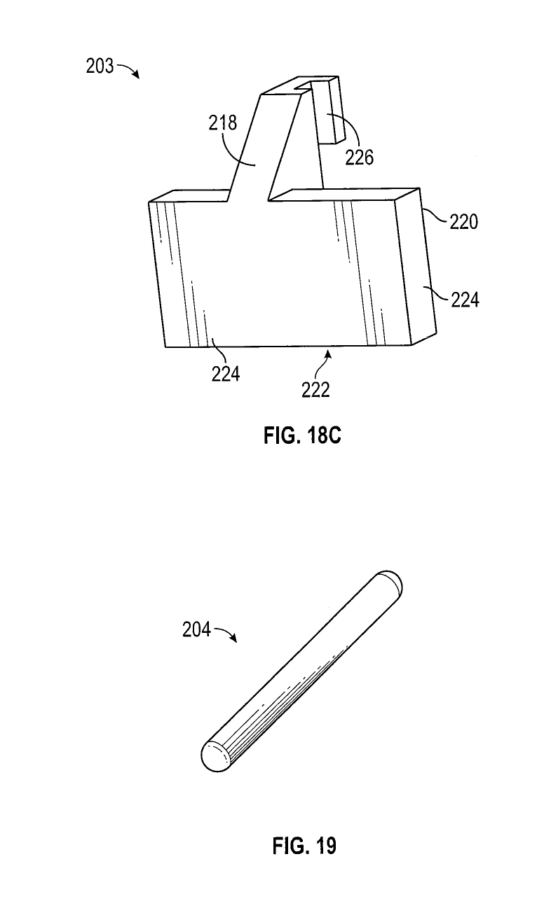

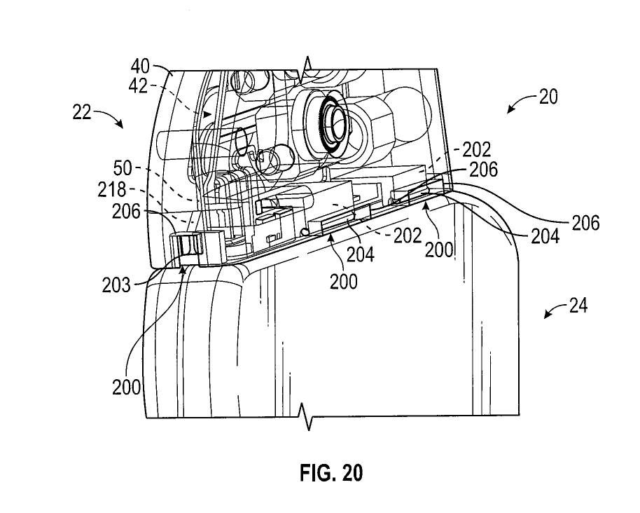

FIG. 17 is an isometric view of a pre-molded header connector assembly 22 with three weld inserts 202 molded into the header 40 of the pre-molded connector assembly. FIG. 18A is an isometric view of a first type of weld insert 202, and FIGS. 18B and 18C are two opposite isometric views of another type of weld insert 203. As can be understood from FIGS. 17 and 18A, in one embodiment, two of the weld inserts 202 are substantially cubical or block shaped. As more clearly depicted in FIG. 18A, the first type of weld insert 202 may have a generally rectangular planar top surface 208, generally rectangular planar side faces 210, and a rectangular central void 212 that extends longitudinally and results in the rectangular planar side faces 210 forming narrow bottom edge faces 214 that extend longitudinally and are spaced apart from each other by the rectangular central void 212. The narrow bottom edge faces 214 are further narrowed by arcuate longitudinally extending grooves 216 that extend the length of the narrow bottom edge faces 214. As discussed below with respect to FIGS. 20-22 and especially FIG. 23, these arcuate longitudinally extending grooves 216 receive in a generally mating fashion weld anchors 204, which are depicted in isometric in FIG. 19. As shown in FIG. 19, a weld anchor 204 has a cylindrical body having a length that is substantially longer than its diameter and may have generally bullnose opposed ends.

As shown can be understood from FIGS. 17, 18B and 18C, in one embodiment, the second type of weld insert 203 may include an arm 218. As more clearly depicted in FIGS. 18B and 18C, the weld insert 203 also includes a rectangular cubical base 220 from which the arm 218 extends. The rectangular cubical base 220 includes a bottom face 222 and rectangular planar side faces 224. The arm 218 includes a vertical slot 226 in which the RF anchor 50 of the connector assembly 42 can be received and welded to the arm 218 of the weld insert 203, as can be understood from FIG. 20, which is an isometric view of the IPG generally opposite from the view depicted in FIG. 16.

In one embodiment, the weld inserts 202, 203 are formed of titanium, titanium alloy, stainless steel, MP35N, 17-4 PH or etc. and pre-molded into the material of the header 40 of the header connector assembly 22 via the same process the connector assembly 42 is pre-molded into the material of the header. The weld anchors 204 are formed of titanium, titanium alloy or etc. and

As can be understood from FIGS. 17 and 20, both types of weld inserts 202, 203 may be employed in a header connector assembly 22. The most forward and middle weld inserts 202 may be of the first type of weld insert 202 depicted in FIG. 18A, and the most rearward weld insert 203 may be of the second type of weld insert 203 depicted in FIGS. 18B and 18C. The most forward and middle weld inserts 202 may be of different sizes with the larger sized weld insert 202 towards the middle of the header connector assembly and a smaller weld insert 202 near the forward or lead receptacle end of the header connector assembly 22. The weld inserts 202, 203 are pre-molded into the pre-molded header connector assembly 22 so as to be imbedded in the material forming the header 40 of the header connector assembly 22. The most forward and middle weld inserts 202 are imbedded in the material of the header 40 such that the narrow bottom edge faces 214 are exposed at the bottom of the header connector assembly 22 and the arcuate longitudinally extending grooves 216 are fully exposed in the weld access windows 206. The most rearward weld insert 202 is imbedded in the material of the header 40 such that the bottom face 222 is exposed at the bottom of the header connector assembly 22, the planar side face 224 opposite the arm 218 is exposed in the adjacent weld access window 206, and the arm 218 projects inwardly to receive and couple with the RF anchor 50. In some embodiments, the second type of weld insert 203 may also have an arcuate groove similar to those grooves 216 of the first type of weld insert 202 so as to receive in a generally mating fashion a weld anchor 204, which is fixedly connected to the housing 24 and also located in the rearward window 206 adjacent the second type of weld insert 203.

In other embodiments, the second type of weld insert 203 is configured to interface with, and be welded to, another type of weld anchor 204 having a different configuration and location other than that depicted in the figures and as discussed above. In one embodiment, as can be understood from FIG. 20, the second type of weld insert 203 is instead an extension from, or coupled to, the housing, and the RF anchor 50, which is imbedded in the material of the housing, is welded to the weld insert 203 to anchor together the housing and header connector assembly.

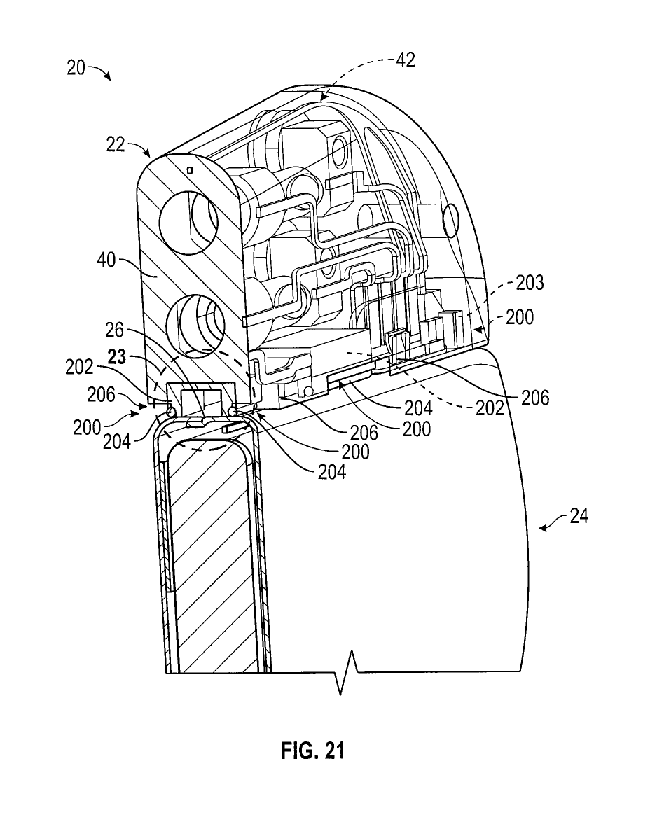

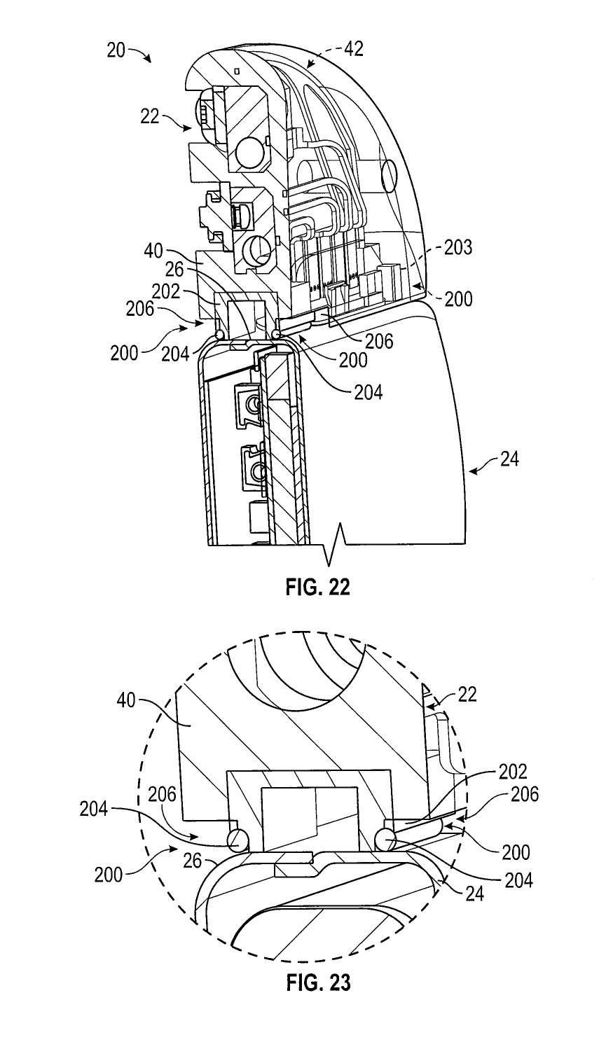

FIGS. 21 and 22 are isometric transverse cross sectional views of the IPG 20 as respectively taken along section lines 21-21 and 22-22 of FIG. 16. As can be understood from FIGS. 20-22, the exposed bottom faces 214 of the weld inserts 202, 203 abut against the top surface 26 of the housing 24 as the header connector assembly 22 is abutted against the housing 24. The weld anchors 204 are received in generally mating abutting arrangement with the respective arcuate longitudinally extending grooves 216 of each first type of weld insert 202, as most clearly depicted in FIG. 23, which is an enlarged transverse cross sectional view of a welded arrangement 200 depicted in FIG. 21. With respect to the second type of weld insert 203, its corresponding weld anchor 204 can simply abut against the weld insert 203 or, if the second type of weld insert 203 has an arcuate longitudinally extending groove 216, its weld anchor 204 can be received similar to the arrangement depicted in FIG. 23. By employing the combination of weld inserts and weld anchors, no direct welding need occur on the hermetically sealed housing 24. As a result, the hermeticity of the housing is maintained.

In one embodiment, the weld anchors are formed, machined or otherwise defined in the material forming the housing, said material being titanium, a titanium alloy, or etc. In another embodiment, the weld anchors are welded or otherwise affixed to the housing prior to the housing having been hermetically sealed or achieving it hermetic certification.

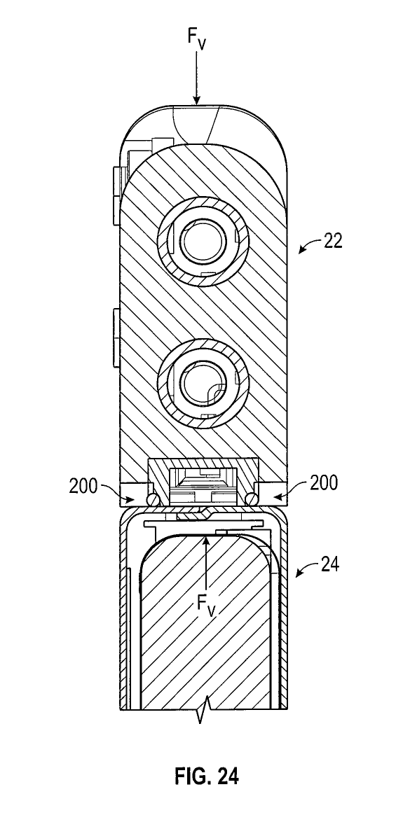

With the weld anchors 204 so positioned against the respective weld inserts 202, 203 and exposed within the respective weld access windows 206, the weld anchors can be resistance or laser welded to the weld inserts. Since the weld inserts 202, 203 are imbedded in the material of the header 40 such that the weld inserts are part of the header connector assembly 22, and since the weld anchors 204 are attached to, or extensions of, the housing 24, the housing 24 and header connector assembly 22 can be clamped together in a compressive pre-load of force Fᵥ, as indicated in FIG. 24, which is a transverse cross section elevation of the IPG similar to that depicted in FIG. 21. With these IPG modules 22, 24 so clamped together, the weld inserts and weld anchors can be welded together, thereby permanently joining/anchoring together these two modules of the IPG 20. The compressive pre-load provides many of the same benefits discussed above with respect to FIG. 14.

The foregoing merely illustrates the principles of the invention. Various modifications and alterations to the described embodiments will be apparent to those skilled in the art in view of the teachings herein. It will thus be appreciated that those skilled in the art will be able to devise numerous systems, arrangements and methods which, although not explicitly shown or described herein, embody the principles of the invention and are thus within the spirit and scope of the present invention. From the above description and drawings, it will be understood by those of ordinary skill in the art that the particular embodiments shown and described are for purposes of illustrations only and are not intended to limit the scope of the present invention. References to details of particular embodiments are not intended to limit the scope of the invention.

1. An implantable pulse generator for administering electrotherapy via an implantable lead, the pulse generator comprising:
a housing comprising a first portion of a cam lock arrangement; and
a header connector assembly comprising a connector assembly, a header enclosing the connector assembly, and a second portion of the cam lock arrangement cam locked with the first portion in anchoring the header connector assembly to the housing.

2. The implantable pulse generator of claim 1, wherein the first portion comprises a pin and the second portion comprises a cam that is cam locked with the pin.

3. The implantable pulse generator of claim 2, wherein the pin comprises a pair of spaced-apart pins, and the cam comprises a cam that is cam locked with the pair of spaced-apart pins.

4. The implantable pulse generator of claim 2, wherein the cam is rotated to become cam locked with the pin.

5. The implantable pulse generator of claim 2, wherein the cam is cam locked and welded with the pin.

6. The implantable pulse generator of claim 2, wherein the pin comprises three pairs of spaced-apart pins, and the cam comprises three cams that are spaced-apart from each other, each cam being cam locked with a respective one of the three pairs of spaced-apart pins.

7. The implantable pulse generator of claim 2, wherein the pin is defined in a material forming the housing.

8. The implantable pulse generator of claim 2, wherein the pin is welded to the housing prior to the housing having been hermetically sealed or certified.

9. The implantable pulse generator of claim 1, wherein the cam lock arrangement compresses together the header connector assembly and the housing.

10. The implantable pulse generator of claim 1, wherein the second portion comprises a pin and the first portion comprises a cam that is cam locked with the pin.

11. An implantable pulse generator for administering electrotherapy via an implantable lead, the pulse generator comprising:
a housing comprising a weld anchor; and
a header connector assembly comprising a connector assembly, a header enclosing the connector assembly, and a weld insert imbedded in a material of the header and welded to the weld anchor in anchoring the header connector assembly to the housing.

12. The implantable pulse generator of claim 11, wherein the weld insert comprises three weld inserts spaced-apart from each other, the weld anchor comprises two pairs of spaced-apart weld anchors, each pair of spaced-apart weld anchors being paired with two of the weld inserts, and the third weld insert is welded to an RF anchor of the connector assembly.

13. The implantable pulse generator of claim 11, wherein the weld anchor is defined in a material forming the housing.

14. The implantable pulse generator of claim 11, wherein the weld anchor is welded to the housing prior to the housing having been hermetically sealed or certified.

15. A method of manufacturing an implantable pulse generator for administering electrotherapy via an implantable lead, the method comprising:
abutting together a housing and a pre-manufactured header connector assembly, the housing comprising a first portion of a cam lock arrangement, and the pre-manufactured header connector assembly comprising a connector assembly, a header enclosing the connector assembly, and a second portion of the cam lock arrangement; and
cam locking together the first and second portions of the cam lock arrangement.

16. The method of claim 15, further comprising applying a compressive pre-load to the housing and pre-manufactured header connector assembly in abutting together the housing and pre-manufactured header connector assembly, the compressive pre-load being in existence prior to and during the cam locking together the first and second portions of the cam lock arrangement.

17. The method of claim 15, wherein the first portion comprises a pin and the second portion comprises a cam that is cam locked with the pin in the course of cam locking together the first and second portions of the cam lock arrangement.

18. The method of claim 17, wherein the pin comprises a pair of spaced-apart pins, and the cam comprises a cam that is cam locked with the pair of spaced-apart pins in the course of cam locking together the first and second portions of the cam lock arrangement.

19. The method of claim 17, wherein the cam is rotated to become cam locked with the pin.

20. The method of claim 17, wherein the cam is cam locked with the pin in the course of cam locking together the first and second portions of the cam lock arrangement followed by welding together the pin and cam.
